(19) United States
(12) Patent Application Publication
(10) Pub. No.: US 2022/0378688 A1
(43) Pub. Date: Dec. 1, 2022

(54) Moisturizing or anti-atopic composition containing fatty acids or fatty acid derivatives

(71) Applicants: DAEBONG LS, LTD, Incheon (KR); UCL CO. LTD., Jeju-si, Jeju-do (KR); JEJU NATIONAL UNIVERSITY INDUSTRY-ACADEMIC COOPERATION FOUNDATION, Jeju-si, Jeju-do (KR)

(72) Inventors: Hyun Sook Yeom, Jeju-si (KR); Won Bo Oh, Seogwipo-si (KR); Hye JA Lee, Seogwipo-si (KR); Ji Hye Kim, Seogwipo-si (KR); Jeong Mi Kim, Jeju-si (KR); Ji Young Moon, Jeju-si (KR); Byoung Kwon Park, Incheon (KR); Jin Oh Park, Seoul (KR); Ji Won Lee, Seoul (KR); Nam Ho Lee, Jeju-si (KR); Jung Eun Kim, Jeju-si (KR)

(21) Appl. No.: 17/775,676

(22) PCT Filed: Nov. 2, 2020
(86) PCT No.: PCT/KR2020/015130

§ 371 Date: May 10, 2022

(30) Foreign Application Priority Data
Nov. 11, 2019 (KR) 10-2019-0143129

(51) Int. Cl. A61K 8/9783; A61K 31/20; A61K 8/36; A61Q 19/00

(52) U.S. Cl. CPC A61K 8/9783; A61K 31/20; A61K 8/361; A61Q 19/005

(57) ABSTRACT

The present disclosure relates to a moisturizing or anti-atopic composition, including a fatty acid or a fatty acid derivative. More specifically, the present disclosure relates to a moisturizing or anti-atopic composition including at least one compound selected from the group consisting of ethyl linoleate, α-linolenic acid, and monolinolein as an active ingredient. The composition for moisturizing or anti-atopic dermatitis, according to the present disclosure, has not only low cytotoxicity but also an anti-inflammatory effect, an increase in the amount of moisturizing factor production, an increase in the amount of skin bather strengthening factor production, and an active effect to inhibit the production of atopic factors, so it can be used in various fields such as beauty for improving skin condition, pharmaceuticals, and food.

### CROSS-REFERENCE TO RELATED APPLICATION

This Application is a Section 371 National Stage Application of International Application No. PCT/KR2020/015130, filed Nov. 2, 2020 and published as WO 2021/096134 A1 on May 20, 2021, in Korean, the contents of which are hereby incorporated by reference in their entirety.

### TECHNICAL FIELD

The present disclosure relates to a moisturizing or anti-atopic composition, including a fatty acid or a fatty acid derivative. More specifically, the present disclosure relates to a moisturizing or anti-atopic composition including at least one compound selected from the group consisting of ethyl linoleate, α-linolenic acid, and monolinolein as an active ingredient.

This application claims priority based on Korea Application No. 10-2019-0143129 filed on Nov. 11, 2019 and all contents disclosed in the specification of the application are incorporated herein by reference.

### BACKGROUND ART

Atopic dermatitis is caused by genetic, environmental, and immunological causes, and abnormalities in the stratum corneum that acts as a bather at the outermost part of the skin, which is an allergic disease that intensifies in dry climates.

The main symptoms of atopic dermatitis are severe itching, dry skin, rash, and scaly skin, which are mainly accompanied by chronic skin inflammation. In particular, if atopic dermatitis causes severe itching in atopic patients, the skin bather collapses due to scratching, thus causing a secondary infection, which can worsen the atopic dermatitis.

The prescription for atopic dermatitis is usually based on medications such as steroids, antihistamines, and antibiotics, etc., but long-term application can cause side effects such as skin weakening, systemic hormone symptoms, and addiction, so it is still required to develop a composition for treating atopic dermatitis that is effective and has no side effects.

Carrots (Daucus carota var. sativa) are a plant of the family Apiaceae, and most of them eat the root part of carrots for food, but the soft leaves and stems taste a combination of celery and water parsley, so it is known to be good to eat raw or with lettuce. In the West, carrots are sold as a whole because the stems and leaves are eaten together, but in Korea, most of the carrots are discarded after harvest because people do not eat the stems of carrots, so physiological activity and ingredient studies on stems and leaves, which are the ground part of carrots, are not known.

### DISCLOSURE

### Technical Problem

An objective of the present disclosure is to provide a composition for moisturizing or anti-atopic including at least one compound selected from the group consisting of ethyl linoleate, α-linolenic acid, and monolinolein as an active ingredient.

### Technical Solution

According to an aspect of this disclosure, a composition for moisturizing or anti-atopic includes at least one compound selected from the group consisting of ethyl linoleate, α-linolenic acid, and monolinolein as an active component.

In this case, the compound may be derived from the stems and leaves part of carrots.

The moisturizing or anti-atopic composition may be a cosmetic composition, a pharmaceutical composition, or a food composition.

The stems and leaves part of carrots, according to an aspect of this disclosure, includes at least one compound selected from the group consisting of ethyl linoleate, α-linolenic acid, and monolinolein as an active component.

Further, the carrot stems and leaves fraction, according to another aspect of the present disclosure, includes at least one compound selected from the group consisting of ethyl linoleate, α-linolenic acid, and monolinolein as an active ingredient.

In this case, the carrot stems and leaves fraction may be a fraction of n-hexane.

### Advantageous Effects

The composition for moisturizing or anti-atopic dermatitis according to the present disclosure has not only low cytotoxicity but also an anti-inflammatory effect, an increase in the amount of moisturizing factor production, an increase in the amount of skin bather strengthening factor production, and an active effect to inhibit the production of atopic factors, so it can be used in various fields such as beauty for improving skin condition, pharmaceuticals, and food.

### BEST MODE

### Mode for Disclosure

Hereinafter, embodiments of the present disclosure will be described in detail with reference to the accompanying drawings so that those of ordinary skilled in the art to which the present disclosure pertains can easily carry out the present disclosure. However, the present disclosure may be embodied in many different forms and is not limited to the embodiments and drawings described herein.

The present inventors have found that although research and utilization of the carrot root part are mainly conducted, research on the activity and components of the stem or leaf, which is the carrot stems and leaves part, is very effective in anti-inflammatory and anti-atopy, thereby completing the present disclosure.

A composition for moisturizing or anti-atopy, according to an aspect of this disclosure, includes at least one compound selected from the group consisting of ethyl linoleate, α-linolenic acid, and monolinolein as an active component.

The α-linolenic acid is a polyunsaturated fatty acid having 18 carbons and 3 double bonds (C18:3) and is known as an omega-3 fatty acid and is found in a variety of seeds and oils, including flaxseed, walnut, chia, hemp and many vegetable oils. In many studies, α-linolenic acid is known to be effective in reducing the risk of arteriosclerosis, reducing the risk of heart disease, high blood pressure, and pneumonia, and in preventing obesity by lowering cholesterol.

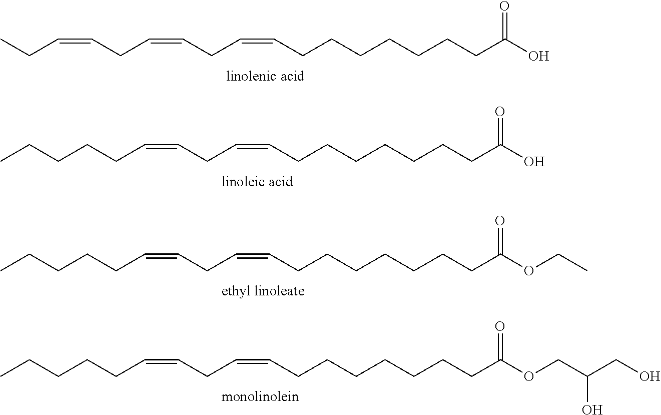

The ethyl linoleate is an ethyl ester of a linoleic acid having two double bonds and refers to one of the essential fatty acids that suppresses the activity of activated oxygen species by bacterial stimulation and suppresses hyperkeratinization induced by deficiency of linoleic acid. It is known in the art that an aqueous ethyl linoleate emulsion can be used for parenteral injection to treat diseases caused by high cholesterol in the blood. It is also known that the administration of ethyl linoleate can improve liver function.

In addition, the monolinolein esterified with one molecule of glycerol and linoleic acid is a polyunsaturated fatty acid used for biosynthesis of arachidonic acid, leukotriene, and thromboxane and is contained in large amounts in lipids, nuts, seeds, and seed oils of the cell membrane. It has been reported that when the monolinolein-deficient diet is provided to experimental animals such as mice, dead skin cells, hair loss, and decreased wound healing ability are induced.

Each of ethyl linoleate, α-linolenic acid, and monolinolein, according to the present disclosure, has not only low cytotoxicity but also an increase in the amount of moisturizing factor production, an increase in the amount of skin bather strengthening factor production, and a remarkably high effect on inhibiting the production of atopic factors, and thus may be used as a composition for moisturizing and anti-atopy, and preferably as a cosmetic composition, a pharmaceutical composition, or a food composition.

In addition, each of the ethyl linoleate, α-linolenic acid, and monolinolein may be separated from vegetable oil or prepared through a simple synthesis process, and more preferably, may be derived from the stems and leaves part of carrots. However, the present disclosure is not limited thereto.

[symbol]

On the other hand, the stems and leaves part of carrots, according to an aspect of this disclosure, includes at least one compound selected from the group consisting of ethyl linoleate, α-linolenic acid, and monolinolein as an active component.

The total content of the compounds may be 0.01 to 5% by weight, preferably 0.5 to 2% by weight, based on the total weight of the carrot stems and leaves extract.

If the total content of the compounds is less than 0.01% by weight, moisturizing and anti-atopic efficacy is not sufficient, and if it exceeds 5% by weight, the difference in efficacy is not large.

The method for producing a carrot stems and leaves extract, according to the present disclosure, includes: drying the stems and leaves part of the carrot; grinding the dried stems and leaves part of the carrot to obtain a fine powder; and extracting the fine powder with 50 to 80% by volume of an aqueous ethanol solution.

At this time, the stems and leaves part of the carrot may be extracted and used without performing a drying step, but it is preferable to include a drying step for long-term storage and stability, such as raw material supply and demand. In order to increase the extraction efficiency of the active component, more preferably, it is to include a grinding step of fine-powdering the structure.

In addition, in order to increase the moisturizing and anti-atopic efficacy, it is preferable to extract the fine powder of the dried carrot above with an aqueous solution of 50 to 80% by volume of ethanol.

When the aqueous ethanol solution contains less than 50% by volume of ethanol, the yield of the carrot stems and leaves extract is low, and when it contains more than 80% by volume of ethanol, the yield of the carrot stems and leaves extract increases. However, it is undesirable because the active efficacy of the active ingredient is relatively low because a lot of other ingredients are contained.

In addition, the aqueous ethanol solution may be extracted using 5 to 15 times the volume of the fine powder compared to the weight.

When the aqueous ethanol solution uses less than 5 times the weight of the fine powder, the extraction efficiency is not high, and when it exceeds 15 times, the extraction efficiency does not increase compared to the amount used, so it is inefficient.

[symbol]

Further, the carrot stems and leaves fraction, according to another aspect of the present disclosure, includes at least one compound selected from the group consisting of ethyl linoleate, α-linolenic acid, and monolinolein as an active ingredient.

The total content of the compounds may be preferably 10 to 30% by weight, based on the total weight of the carrot stems and leaves fraction.

If the total content of the compounds is less than 10% by weight, moisturizing and anti-atopic efficacy is not sufficient, and if it exceeds 30% by weight, cytotoxicity may occur.

The method for producing a carrot stems and leaves fraction according to the present disclosure includes: extracting and concentrating the stems and leaves part of carrots with 50 to 80% by volume of an aqueous ethanol solution to obtain an extract; suspending the obtained extract in distilled water to prepare a suspension; and fractionating and concentrating the suspension with an organic solvent to obtain a fraction.

In the step of obtaining an extract, when the aqueous ethanol solution contains less than 50% by volume of ethanol, the yield of the carrot stems and leaves extract is low, and when it contains more than 80% by volume of ethanol, the yield of the carrot stems and leaves extract increases. However, it is undesirable because the active efficacy of the active ingredient is relatively low because a lot of other ingredients are contained.

In addition, in the step of preparing the suspension, it is preferable to use 10 to 20 times the volume of distilled water relative to the mass of the extract. If the volume is less than 10 times the mass of the extract, the extract is not evenly suspended, and clumps occur, and if the volume exceeds 20 times, the use of water during extraction and fractionation increases, which is inefficient.

The organic solvent is preferably at least one organic solvent selected from the group consisting of n-alkane, ethyl acetate, methylene chloride, and n-butanol having 4 to 10 carbon atoms. More preferably, it may be any one selected from n-pentane, n-hexane, and n-heptane.

The organic solvent for the fractionation is preferably used in an amount of 0.5 to 1.5 times the volume of distilled water used in the step of preparing the suspension. When less than 0.5 times the volume is used, the extraction efficiency is lowered. When the volume is exceeded 1.5 times, the extraction efficiency is lowered compared to the amount used, which is inefficient.

As described above, each ethyl linoleate, α-linolenic acid, and monolinolein has not only low cytotoxicity but also an increase in the amount of moisturizing factor production, an increase in the amount of skin bather strengthening factor production, and a remarkably high effect on inhibiting the production of atopic factors, and thus may be used as a composition for moisturizing and anti-atopy, and preferably as a cosmetic composition, a pharmaceutical composition, or a food composition.

In addition, the carrot stems and leaves extract and fraction of the present disclosure using carrot stems and leaves parts includes more than one type of compound selected from the group consisting of ethyl linoleate, α-linolenic acid, and monolinolein as an active ingredient. It is eco-friendly because it can be usefully used as a cosmetic composition, pharmaceutical composition, and food composition with excellent skin bather reinforcement, moisturization, and anti-atopy effects, and discarded the stems and leaves part of carrots can be used as active materials.

[symbol]

Hereinafter, the present disclosure will be described in more detail through specific examples. The following examples are intended to illustrate the present disclosure, but the present disclosure is not limited by the following examples.

[symbol]

### PREPARATION EXAMPLE 1. PREPARATION OF CARROT STEMS AND LEAVES EXTRACT

After washing the stems and leaves part of carrots with distilled water, dried and then pulverized with a blender to obtain a fine powder sample. The solvent in which ethanol and purified water were mixed at a ratio of 7:3 was added in a volume amount of about 10 times based on the weight of each sample of the fine powder (200 g) of the carrot stems and leaves part, and then extraction was performed twice. After performing the extraction, the resultant extract was filtered through a 400 mesh filter cloth, and then the resulting filtrate was concentrated 100% using a vacuum concentrator to obtain an extract of carrot stems and leaves (65 g), which was used for the test.

[symbol]

### PREPARATION EXAMPLE 2. SEPARATION OF COMPOUNDS FROM EXTRACTS

The carrot stems and leaves extract (60 g) prepared in Preparation Example 1 was suspended in 1 L of distilled water, 1 L of n-hexane was added and mixed vigorously, and the n-hexane layer was fractionated using a separatory funnel and fractionated under reduced pressure to obtain an n-hexane fraction (3 g). Vacuum liquid chromatography (VLC) was performed to subdivide this fraction according to polarity, and 15 fractions were obtained by dissolving 200 mL each by increasing the solvent polarity of n-Hexane-EtOAc (0-50%) by 3 or 5%, respectively (V1 to V15). Among these fractions, V3 (384 mg) was Compound 1, 74.5 mg and 96.8 mg of Compound 2 were obtained in V7 (491.6 mg) and V8, respectively, and in V15 (68.8 mg), Sephadex LH-20 column (CHCl₃: MeOH=15:1) to obtain Compound 3 (48.3 mg), which was used for the test. For each compound, Compound 1 (ethyl linoleate), Compound 2 (α-linolenic acid), and Compound 3 (Monolinolein) were identified using nuclear magnetic resonance (NMR).

[symbol]

### EXPERIMENTAL EXAMPLE 1. CELL CULTURE

### (1) Macrophage Culture

Macrophages RAW264.7 cells were distributed from American Type Cell Culture (ATCC) and Dulbecco's Modified Eagle's Medium (DMEM) medium containing 100 units/ml of penicillin-streptomycin and 10% by volume of fetal bovine serum (FBS) was used and cultured at 37° C. and 5% CO₂ incubator. Subculture was performed at intervals of 2 to 3 days.

### (2) Human Epidermal Keratinocyte Culture

HaCaT cells, which are human epidermal keratinocytes, were distributed from Dr. C. G. Hyun (Jeju National University, Korea) and Dulbecco's Modified Eagle's Medium (DMEM) medium containing 100 units/ml of penicillin-streptomycin and 10% by volume of fetal bovine serum (FBS) was used and cultured at 37° C. and 5% CO₂ incubator. Subculture was performed at intervals of 3 to 4 days.

[symbol]

### EXPERIMENTAL EXAMPLE 2. CYTOTOXICITY ASSESSMENT

### (1) MTT Assay

MTT assay is a representative method for measuring cell viability using the principle that MTT (3-(4,5-dimethylthiazol-2-yl-2,5-diphenyltetrazolium bromide) reacts with dehydrogenase of living cells to generate purple formazan.

RAW264.7 cells were aliquoted in a 96-well plate at 1.5×105 cells/mL using DMEM medium supplemented with 10% by volume of FBS to check cytotoxicity and then cultured at 37° C., 5% CO₂ condition for 18 hours. The extract to be evaluated was treated by exchanging the cultured RAW264.7 cells with DMEM containing 0.1 μg/mL of LPS. After that, EZ-cytox was added to each well and reacted for 3 hours at 37° C. and 5% CO₂, and absorbance was measured at 570 nm using a microplate reader. The average absorbance value for each sample group was obtained, and the cell viability was evaluated by comparing it with the absorbance value of the control group.

### (2) EZ-Cytox Assay

The EZ-cytox assay is a representative method for measuring cell viability using the principle that water solution tetrazolium salt (WST) reacts with dehydrogenase of living cells to generate orange water-soluble formazan.

HaCaT cells were aliquoted in a 96-well plate at 1.0×104 cells/mL using DMEM medium supplemented with 10% by volume of FBS to check cytotoxicity and then cultured at 37° C., 5% CO₂ condition for 18 hours. The cultured HaCaT cells were exchanged with serum-free DMEM to treat the extract to be evaluated. After that, EZ-cytox was added to each well and reacted for 30 minutes at 37° C. and 5% CO₂, and absorbance was measured at 450 nm using a microplate reader. The average absorbance value for each sample group was obtained, and the cell viability was evaluated by comparing it with the absorbance value of the control group.

Table 1 below is showing the evaluation of the cell growth rate of macrophages. As shown in Table 1, no cytotoxicity was observed in macrophages in all samples used for the test.

TABLE 1

| | Cytotoxicity | |
|---|---|---|
| | Treatment | |
| | concentration | RAW264.7 Cell |
| Sample | (μg/mL) | growth rate (%) |
| Untreated group | — | 110.0 ± 2.1 |
| Stimulant (LPS) | 0.1 | 100.0 ± 6.0 |
| Carrot stems and leaves | 100 | 101.0 ± 4.1 |
| extract | 200 | 98.7 ± 5.9 |
| (Example 1) | 300 | 105.7 ± 4.0 |
| | 400 | 120.1 ± 5.8 |
| Carrot roots extract | 100 | 98.9 ± 5.7 |
| (Comparative Example) | 200 | 91.0 ± 3.3 |
| | 300 | 92.3 ± 1.8 |
| | 400 | 92.7 ± 0.3 |

Table 2 below shows the evaluation of the cell growth rate of human epidermal keratinocytes. As shown in Table 2, no cytotoxicity was observed in human epidermal keratinocytes in all samples used for the test.

TABLE 2

| | Cytotoxicity | | |
|---|---|---|---|
| | Treatment | | HaCaT cell |
| Sample | concentration | | growth rate (%) |
| Untreated group | — | | 100.0 ± 3.5 |
| Carrot roots extract | 12.5 | μg/mL | 98.6 ± 1.6 |
| (Comparative | 25 | μg/mL | 101.0 ± 3.3 |
| Example) | 50 | μg/mL | 108.3 ± 4.4 |
| | 100 | μg/mL | 95.6 ± 7.1 |
| Carrot stems and | 12.5 | μg/mL | 105.0 ± 1.2 |
| leaves extract | 25 | μg/mL | 101.9 ± 3.9 |
| (Example 1) | 50 | μg/mL | 96.9 ± 2.6 |
| | 100 | μg/mL | 100.3 ± 4.1 |
| Monolinolein | 12.5 | μM | 106.0 ± 2.6 |
| (Example 2) | 25 | μM | 102.2 ± 1.0 |
| | 50 | μM | 101.5 ± 1.7 |
| | 100 | μM | 122.6 ± 2.1 |
| Ethyl linoleate | 12.5 | μM | 92.5 ± 3.0 |
| (Example 3) | 25 | μM | 92.3 ± 2.0 |
| | 50 | μM | 88.9 ± 2.3 |
| | 100 | μM | 94.1 ± 3.0 |
| α-Linolenic acid | 12.5 | μM | 97.8 ± 3.6 |
| (Example 4) | 25 | μM | 96.1 ± 4.4 |
| | 50 | μM | 92.1 ± 3.9 |
| | 100 | μM | 106.9 ± 1.0 |
| Retinoic acid | 10 | μM | 103.8 ± 2.1 |

(Control group)

[symbol]

### EXPERIMENTAL EXAMPLE 3. CONFIRMATION OF THE EFFECT OF INCREASING THE PRODUCTION OF HYALURONIC ACID, A MOISTURIZING FACTOR

HaCaT cells were aliquoted in a 24-well plate at 1.0×10⁵ cells/mL and cultured at 37° C., 5% CO₂ conditions for 18 hours. It was replaced with a serum-free DMEM medium, and the samples in Table 2 were treated and cultured for 24 hours. After that, the culture medium was removed, centrifuged at 15,000 rpm for 5 minutes, and the supernatant was removed and stored frozen (−20° C.) until quantification. As a control group, a sample treated with retinoic acid (RA) at a concentration of 10 μM was used. Enzyme-Linked Immunosorbent Assay (ELISA) was performed using a hyaluronic acid ELISA kit (Elabscience Biotechnology Co., Ltd) and the method provided by the manufacturer.

Table 3 below shows the results of measuring the hyaluronic acid production of the carrot stems and leaves extract and the carrot root extract. Table 4 below shows the measurement results of the hyaluronic acid production of the compounds isolated from the compounds the carrot stems and leaves extract.

TABLE 3

| | Treatment | | HA |
|---|---|---|---|
| Sample | concentration | | production (%) |
| Untreated group | — | | 100.0 ± 0.31 |
| Carrot stems and | 12.5 | μg/mL | 101.6 ± 0.11 |
| leaves extract | 25 | μg/mL | 115.2 ± 0.07 |
| (Example 1) | 50 | μg/mL | 118.6 ± 0.07 |
| Carrot roots extract | 12.5 | μg/mL | 98.8 ± 0.11 |
| (Comparative | 25 | μg/mL | 99.8 ± 0.05 |
| Example) | 50 | μg/mL | 120.1 ± 0.12 |
| Retinoic acid | 10 | μM | 153.1 ± 0.14 |

(Control group)

TABLE 4

| | Treatment | HA |
|---|---|---|
| | concentration (μM) | production (%) |
| Untreated group | — | 100.0 ± 0.36 |
| Monolinolein | 25. | 102.7 ± 0.14 |
| (Example 2) | 50. | 113.7 ± 0.14 |
| | 100. | 114.4 ± 0.05 |
| Ethyl linoleate | 25. | 92.7 ± 0.27 |
| (Example 3) | 50. | 85.5 ± 0.27 |
| | 100. | 89.5 ± 0.07 |
| α-Linolenic acid | 25. | 105.0 ± 0.06 |
| (Example 4) | 50. | 110.5 ± 0.02 |
| | 100. | 116.3 ± 0.26 |
| Retinoic acid | 10. | 100.0 ± 0.36 |
| (Control group) | | |

Referring to Table 3, it was confirmed that the carrot stems and leaves extract increased the amount of hyaluronic acid production more than that of the carrot roots extract at a low concentration. Comparing Table 3 and Table 4, the stems and leaves and roots carrot extracts showed hyaluronic acid production of 118.6 and 120.1 at a treatment concentration of 50 μg/mL, whereas each of Compound 1 (Ethyl linoleate), Compound 2 (α-linolenic acid), and compound 3 (Monolinolein) shows a production amount of hyaluronic acid of 85.5 to 113.7 even at a relatively low treatment concentration of 50 μM. As a result, it was confirmed that the production amount of hyaluronic acid was significantly increased compared to the extracts, and in particular, it was confirmed that compounds 2 and 3 had a greater effect than compound 1.

[symbol]

### EXPERIMENTAL EXAMPLE 4. CONFIRMATION OF SKIN BATHER STRENGTHENING

HaCaT cells were aliquoted in a 24-well plate at 1.0×105 cells/mL and cultured at 37° C., 5% CO₂ conditions for 18 hours. It was exchanged with serum-free DMEM, and the carrot stems and leaves extracts and roots extracts of the samples in Table 2 were treated and cultured for 24 hours. After removing the culture medium and washing with PBS for each group, PBS is containing no drug that may affect protein quantification was treated. The protein was collected after lysing the cells by repeating low-temperature and room-temperature incubation. As a control group, a sample treated with retinoic acid (RA) at a concentration of 10 μM was used. Quantification was performed using the Filaggrin-ELISA kit (Elabscience Biotechnology Co., Ltd) and was performed by the method provided by the manufacturer.

Table 5 below is the measurement result of the amount of filaggrin production of the carrot stems and leaves extract and the carrot roots extract, and it can be confirmed that the carrot stems and leaves extract increases the amount of filaggrin production more than carrot roots extract.

TABLE 5

| | Treatment | | FLG |
|---|---|---|---|
| Sample | concentration | | production (%) |
| Untreated group | — | | 100.0 ± 0.24 |
| Carrot stems and | 12.5 | μg/mL | 99.7 ± 0.08 |
| leaves extract | 25 | μg/mL | 100.6 ± 0.15 |
| (Example 1) | 50 | μg/mL | 127.6 ± 0.13 |
| Carrot roots extract | 12.5 | μg/mL | 93.0 ± 0.1 |
| (Comparative | 25 | μg/mL | 98.8 ± 0.04 |
| Example) | 50 | μg/mL | 100.4 ± 0.01 |
| Retinoic acid | 10 | μM | 157.5 ± 0.13 |

(Control group)

[symbol]

### EXPERIMENTAL EXAMPLE 5. MEASUREMENT OF TARC PRODUCTION INHIBITORY ACTIVITY, AN ATOPIC FACTOR

In order to confirm the atopic treatment effect on the carrot stems and leaves extract according to the present disclosure, atopic thymus and activation regulated chemokine (TARC) was analyzed.

HaCaT cells were aliquoted in a 24-well plate at 1.5×10⁵ cells/mL and cultured at 37° C., 5% CO₂ conditions for 18 hours. It was exchanged with serum-free DMEM medium, and the samples in Table 2 and interferon-γ (IFN-γ, 10 ng/mL) were treated together and incubated for a certain period of time. Then, the atopic chemokine production content of the supernatant obtained by centrifugation of the culture medium (12,000 rpm, 3 min) was measured. All samples were stored frozen (−20° C.) until quantification. For TARC content, a human enzyme-linked immunosorbent assay (ELISA) kit (R&D Systems Inc., Minneapolis, Minn., USA) was used, and the r² value of the standard curve for the standard was 0.99 or more.

Table 6 below shows the results of measuring the TARC production of the carrot stems and leaves extract and the carrot roots extract, and Table 7 below shows the measurement results of the TARC production of the compounds isolated from the carrot stems and leaves extract.

TABLE 6

| | Treatment | | |
|---|---|---|---|
| | concentration | | TARC |
| | μg/mL) | | production (%) |
| Untreated group | — | — | 20.0 ± 0.15 |
| IFN (30 ng/ml) + TNF (30 | — | + | 100 ± 0.33 |
| ng/ml) | | | |
| Carrot stems and leaves extract | 25. | + | 38.4 ± 0.18 |
| (Example 1) | 50. | + | 28.9 ± 0.16 |
| | 100. | + | 11.6 ± 0.07 |
| Carrot roots extract | 25. | + | 64.5 ± 0.18 |
| (Comparative Example) | 50. | + | 62.4 ± 0.27 |
| | 100. | + | 48.0 ± 0.16 |

TABLE 7

| | Treatment | | TARC |
|---|---|---|---|
| | concentration | | production |
| | (μM) | | (%) |
| Untreated group | — | — | 18.6 ± 0.47 |
| IFN (30 ng/ml) + TNF (30 | — | + | 100.0 ± 1.07 |
| ng/ml) | | | |
| Monolinolein | 12.5. | + | 75.2 ± 0.07 |
| (Example 2) | 25. | + | 37.7 ± 1.59 |
| | 50. | + | 30.9 ± 1.97 |
| Ethyl linoleate | 12.5. | + | 78.3 ± 0.10 |
| (Example 3) | 25. | + | 53.8 ± 0.16 |
| | 50. | + | 42 ± 0.81 |
| α-Linolenic acid | 12.5. | + | 72.6 ± 0.03 |
| (Example 4) | 25. | + | 45.6 ± 0.51 |
| | 50. | + | 35.7 ± 1.44 |

Referring to Table 6, it can be seen that the carrot stems and leaves extract has a higher inhibition rate of atopic chemokine production than the roots carrot extract. Comparing Tables 6 and 7, the carrot stems and leaves extract showed an inhibition rate of atopic chemokine production of 28.9 at a treatment concentration of 50 μg/mL. However, it can be seen that compound 1 (Ethyl linoleate), compound 2 (α-linolenic acid), and compound 3 (Monolinolein) all show inhibition of atopic chemokine production of 30 to 42 even at a relatively low treatment concentration of 50 μM.

[symbol]

### EXPERIMENTAL EXAMPLE 6. CONFIRMATION OF NITRIC OXIDE (NO) PRODUCTION INHIBITORY ACTIVITY AS AN ANTI-INFLAMMATORY EFFECT

RAW264.7 cells were aliquoted in a 24-well plate at 1.5×10⁵ cells/mL and cultured at 37° C., 5% CO₂ conditions for 18 hours. It was replaced with a DMEM medium supplemented with 10% by volume of FBS, and the samples in Table 1 were treated and cultured for 24 hours. 100 μl of cell culture supernatant was recovered in a 96 well plate, 100 μl of Griess reagent was added, and reacted at room temperature for 10 minutes. Then, the absorbance was measured at 540 nm using a microplate reader.

Table 8 below is the measurement of the NO production inhibition amount of the carrot stems and leaves extract and the carrot roots extract, and it can be confirmed that the carrot stems and leaves extract more inhibits NO generation than the carrot roots extract.

TABLE 8

| | Treatment | | |
|---|---|---|---|
| | concentration | | NO |
| | (μg/mL) | | production (%) |
| Untreated group | — | — | 2.8 ± 2.25 |
| Stimulant (LPS) | — | + | 100.0 ± 3.5 |
| Carrot stems and | 100. | + | 90.6 ± 2.05 |
| leaves extract | 200. | + | 75.6 ± 4.00 |
| (Example 1) | 300. | + | 55.9 ± 2.11 |
| | 400. | + | 43.4 ± 6.43 |
| Carrot roots extract | 100. | + | 104 ± 7.89 |
| (Comparative | 200. | + | 102.5 ± 4.99 |
| Example) | 300. | + | 93.0 ± 2.00 |
| | 400. | + | 92.0 ± 0.12 |

Overall, the present inventors confirmed that the carrot stems and leaves extract had low cytotoxicity as well as anti-inflammatory effects, increased production of moisturizing factors, increased production of skin bather strengthening factors, and inhibitory activity of atopic factor production. The carrot stems and leaves extract of the present disclosure can be variously used in the fields of beauty, pharmaceuticals, and food for improving skin conditions.

[symbol]

Hereinafter, the present disclosure will be described in more detail through specific examples. The following examples are intended to illustrate the present disclosure, but the present disclosure is not limited by the following examples.

[symbol]

### PREPARATION EXAMPLE 1. SOFT LOTION MANUFACTURING

0.1% by weight of ethyl linoleate,

0.1% by weight of α-linolenic acid,

0.1% by weight of monolinolein,

2.0% by weight of butylene glycol,

2.0% by weight of propylene glycol,

0.1% by weight of acrylate/C10 to C30 alkyl acrylate cross-polymer,

0.4% by weight of polysorbate 80,

0.1% by weight of argininic acid,

0.1% by weight of xanthan gum,

1.0% by weight of hyaluronic acid,

appropriate amounts of preservatives, colors, and fragrances,

purified water up to 100.

[symbol]

### PREPARATION EXAMPLE 2. CREAM MANUFACTURING

0.1% by weight of ethyl linoleate,

0.1% by weight of α-linolenic acid,

0.1% by weight of monolinolein,

5.0% by weight of beta-1,3-glucan,

1.5% by weight of polysorbate 80,

5.0% by weight of squalane,

5.0% by weight of glycerin,

3.0% by weight of butylene glycol,

3.0% by weight of propylene glycol,

1.0% by weight of cetearyl olivate/sorbitan olivate,

appropriate amounts of preservatives, colors, and fragrances,

purified water up to 100.

[symbol]

### PREPARATION EXAMPLE 3. MANUFACTURING OF EXTERNAL PREPARATIONS FOR SKIN

0.1% BY WEIGHT OF ETHYL LINOLEATE,

0.1% by weight of α-linolenic acid,

0.1% by weight of monolinolein,

5.0% by weight of beta-1,3-glucan,

5.0% by weight of polysorbate 80,

2.0% by weight of PEG 60,

5.0% by weight of shea butter,

5.0% by weight of squalane,

10.0% by weight of glycerin,

10.0% by weight of propylene glycol,

1.0% by weight of cetearyl olivate/sorbitan olivate,

appropriate amounts of preservatives, colors, and fragrances,

purified water up to 100.

[symbol]

Although the composition ratio is prepared by mixing relatively suitable components in a preferred embodiment, the mixing ratio may be arbitrarily modified according to regional and ethnic preferences such as demanding class, demanding country, and use.

[symbol]

The above description is merely an example of the present disclosure, and it will be understood by those skilled in the art to which the present disclosure belongs that the present disclosure may be implemented in a deformed form without departing from the essential characteristics of the present disclosure. Therefore, the disclosed embodiments and experimental examples should be considered from an illustrative rather than a restrictive point of view. The scope of the present disclosure is indicated in the claims rather than the foregoing description, and all differences within the scope equivalent thereto should be construed as being included in the present disclosure.

1. A moisturizing or anti-atopic composition comprising at least one compound selected from the group consisting of ethyl linoleate, α-linolenic acid, and monolinolein, as an active ingredient.

2. The composition of claim 1, wherein the compound is derived from the stems and leaves part of carrots.

3. The composition of claim 1, wherein the moisturizing or anti-atopic composition is a cosmetic composition, a pharmaceutical composition, or a food composition.

4. A carrot stems and leaves extract comprising one or more compounds selected from the group consisting of ethyl linoleate, α-linolenic acid, and monolinolein as active ingredients.

5. A carrot stems and leaves fraction comprising one or more compounds selected from the group consisting of ethyl linoleate, α-linolenic acid, and monolinolein as active ingredients.

6. The fraction of claim 5, wherein the carrot stems and leaves fraction is n-hexane fraction.
